(19) United States
(12) Patent Application Publication
(10) Pub. No.: US 2023/0021945 A1
(43) Pub. Date: Jan. 26, 2023

(54) SYSTEMS AND METHODS FOR DYNAMICALLY GENERATING EXERCISE PLAYLIST

(71) Applicant: Rovi Guides, Inc., San Jose, CA (US)

(72) Inventors: Padmassri Chandrashekar, Bangalore (IN); Daina Emmanuel, Bangalore (IN); Reda Harb, Bellevue, WA (US); Akshay Chetan Shah, Mumbai (IN)

(21) Appl. No.: 17/374,643

(22) Filed: Jul. 13, 2021

(51) Int. Cl. A63B 71/06; A63B 24/00

(52) U.S. Cl. CPC A63B 71/0622; A63B 24/0062; A63B 24/0075; G06K 9/00342

(57) ABSTRACT

Systems and methods are described for generating for presentation to a user at least one modified segment corresponding to a physical exercise depicted during presentation of a media asset to a user. The media asset may comprise multiple segments and one or more exercises, where each respective exercise corresponds to one or more segments, and input may be received from one or more sensors during presentation of the media asset, where the input is related to the user. Based on the received input, a determination may be made that an alternate version of an exercise corresponding to at least one particular segment of the multiple segments should be provided instead of a version of the exercise scheduled to be provided. The at least one particular segment may be modified to correspond to the alternate version of the exercise, and the at least one modified particular segment may be generated for presentation.

### BACKGROUND

This disclosure is directed to providing an alternate version of an exercise depicted in a media asset. Specifically, techniques are disclosed for modifying at least one particular segment corresponding to an exercise to correspond to an alternate version of the exercise and generating for presentation to the user the modified at least one particular segment.

### SUMMARY

Many users have become accustomed to participating in live or prerecorded (on-demand) exercise videos provided over the Internet, where an instructor guides the user through various types of workouts, such as group workout classes intended for consumption by a large audience. Indeed, many users have canceled their gym memberships in favor of the convenience and privacy of exercising in one's own home at any time of day that is desired. However, not all users are at the same fitness level, and providing the same static workout class to each user can be problematic for users for which the class is too easy, as well as for users for which the class is too difficult. In one approach, information provided by a user as well as biometric data can be utilized to optimize a workout session. However, this approach merely suggests a completely different series of exercises to the user, and fails to personalize each individual exercise of a workout class to the characteristics of the user measured in real time, and fails to adequately balance the desire of the user to participate in workouts of a certain type with the current physical abilities of the user.

To overcome these problems, systems and methods are provided herein for providing an alternate version of an exercise depicted in a media asset. A media asset comprising a plurality of segments may be generated for presentation to a user, where the media asset when generated for presentation depicts one or more exercises, and each respective exercise corresponds to one or more segments of the plurality of segments. For example, a particular segment may correspond to a portion (e.g., 7 seconds) of an exercise (e.g., a plank exercise depicted for a total of one minute in the media asset). Input may be received from one or more sensors during the presentation of the media asset, where the input is related to the user. Based on the received input, a determination may be made that an alternate version of an exercise corresponding to at least one particular segment should be provided instead of a version of the exercise scheduled to be provided. The at least one particular segment may be modified to correspond to the alternate version of the exercise, and the at least one modified particular segment may be generated for presentation to the user.

Such aspects enable a system to provide a personalized playlist of exercises to a user based on real-time sensor data provided by one or more sensors monitoring a user performing the exercises depicted in the media asset. Based on the measured response (e.g., biometric data) of the user to the exercises of the media asset and/or other factors (e.g., spatial constraints of the workout space of the user, a determination whether the form or posture of the user during the workout is proper, nutrition and lifestyle characteristics of the user, nutrition habits of the user), a playlist of exercises may be updated in real time to maximize the benefit of each exercise for the user. For example, if the system detects that a user is performing poorly during a particular exercise, the next exercise of the media asset may be lowered in intensity (e.g., modified from a standard plank in the “Advanced” playlist to a modified plank in the “Intermediate” playlist) while still adhering to the intent of the workout. On the other hand, if the performance of the user improves during the modified workout, the exercise playlist may revert to the initially schedule playlist (e.g., back to the “Advanced” playlist) to account for the improvement in performance of the user. That is, the system may navigate various alternative logical paths for a given media asset based on the performance of the user, to dynamically populate the exercise playlist in a manner that is personalized to the user.

In some embodiments, at least one of the one or more sensors is associated with exercise equipment used during the exercise. The system may provide a recommendation for adjusting of, or cause automatic adjustment of, the exercise equipment (e.g., resistance on an exercise bike or an amount of weight for an adjustable dumbbell) based on the input from the one or more sensors.

In some aspects of this disclosure, the system may receive user information from a plurality of users, where the user information is associated with exercise sessions that respective users of the plurality of users participated in. Based on the received user information, a particular exercise of the exercise sessions associated with a user injury may be identified, and an alternate version of the exercise may be provided based on the received input from the one or more sensors and the identified exercise associated with the user injury.

In some embodiments, determining that the alternate version of the exercise corresponding to the at least one particular segment should be provided comprises determining, based on the input from one or more sensors, that an additional user, in addition to the user, is consuming the media asset, and determining a current state of the user and the additional user based on the input from one or more sensors. The alternate version of the exercise may correspond to a joint exercise for each of the user and the additional user, and a recommendation may be provided, based on the determined current states of the user and the additional user, that either the user or the additional user perform a higher intensity portion of the joint exercise than the other of the user or the additional user.

In some aspects of this disclosure, each respective exercise may be associated with a plurality of versions of the exercise corresponding to different intensity levels of the exercise, and each version of the exercise may be tagged with an indication of one or more attributes. The system may be configured to determine, based on the received input, that the alternate version of the exercise corresponding to the at least one particular segment should be provided by determining, based on the input from the one or more sensors, attributes associated with activity of the user during the presentation of the particular segment, and comparing the determined attributes to the tagged attributes to determine a version of the exercise having attributes matching the determined attributes. The at least one particular segment of the media asset may be modified to correspond to the alternate version of the exercise based on the comparing.

In some embodiments, the received input corresponds to biometric data of the user, and determining, based on the received input, that the alternate version of the exercise corresponding to the at least one particular segment should be generated for presentation comprises determining, based on the received biometric data, a current state of the user during a current segment of the media asset and determining, based on the current state of the user, that the alternate version of the exercise should be provided during a next segment of the media asset following the current segment.

In some aspects of this disclosure, each respective exercise is associated with a plurality of versions of the exercise corresponding to different intensity levels of the exercise. The system may be configured to determine, based on the current state of the user, that the alternate version of the exercise should be provided during the next segment of the media asset following the current segment by determining that the received biometric data of the user is outside a predefined range, and, in response to determining that the received biometric data of the user is outside the predefined range, causing the alternate version of the exercise to be a lighter intensity version compared to the version of the exercise scheduled to be provided.

In some embodiments, the system may be configured to receive nutrient information related to nutrients consumed by the user within a predefined period of time from a current time, where the current state of the user during the current segment of the media asset may be determined based on the received biometric data of the user and the received nutrient information.

In some aspects of this disclosure, the system may be configured to identify, based on the received input, a posture or form of the user during the current segment; determine whether the identified posture or form of the user is proper; where determining the current state of the user during the current segment of the media asset is based on the received biometric data of the user and whether the identified posture or form of the user is proper.

In some embodiments, the biometric data of the user may be received during a warm-up segment of the media asset used to assess the physical abilities of the user.

### BRIEF DESCRIPTION OF THE DRAWINGS

The above and other objects and advantages of the present disclosure will be apparent upon consideration of the following detailed description, taken in conjunction with the accompanying drawings, in which like reference characters refer to like parts throughout, and in which:

FIG. 1 shows an illustrative environment in which an alternate version of an exercise depicted in a media asset may be provided to a user, in accordance with some embodiments of this disclosure;

FIG. 2 shows a block diagram of an illustrative system for providing an alternate version of an exercise depicted in a media asset, in accordance with some embodiments of this disclosure;

FIG. 3 shows a block diagram of an illustrative technique for identifying posture or form of a user during an exercise, in accordance with some embodiments of this disclosure;

FIG. 4 shows an illustrative environment in which an alternate version of an exercise depicted in a media asset may be provided to multiple users in a joint exercise, in accordance with some embodiments of this disclosure;

FIG. 5 shows a block diagram of an illustrative media device in a system for providing an alternate version of an exercise depicted in a media asset, in accordance with some embodiments of this disclosure;

FIG. 6 shows a block diagram of an illustrative media system for providing an alternate version of an exercise depicted in a media asset, in accordance with some embodiments of this disclosure;

FIG. 7 is a flowchart of a detailed illustrative process for providing an alternate version of an exercise depicted in a media asset, in accordance with some embodiments of this disclosure; and

FIG. 8 is a flowchart of a detailed illustrative process for determining whether to provide an alternate version of an exercise depicted in a media asset, in accordance with some embodiments of this disclosure.

### DETAILED DESCRIPTION

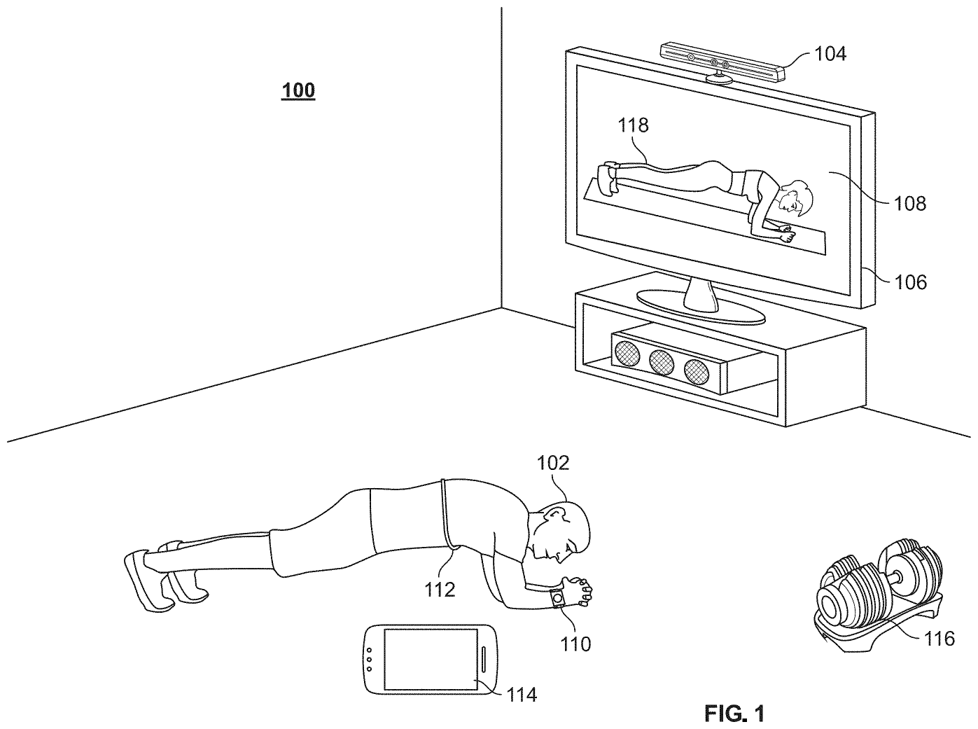

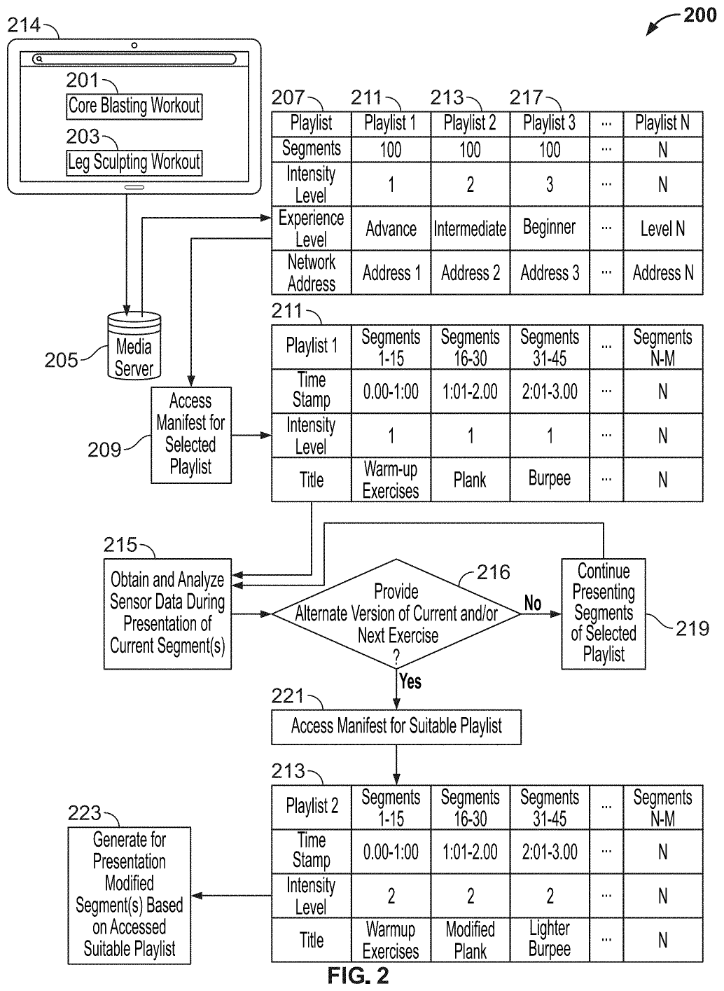

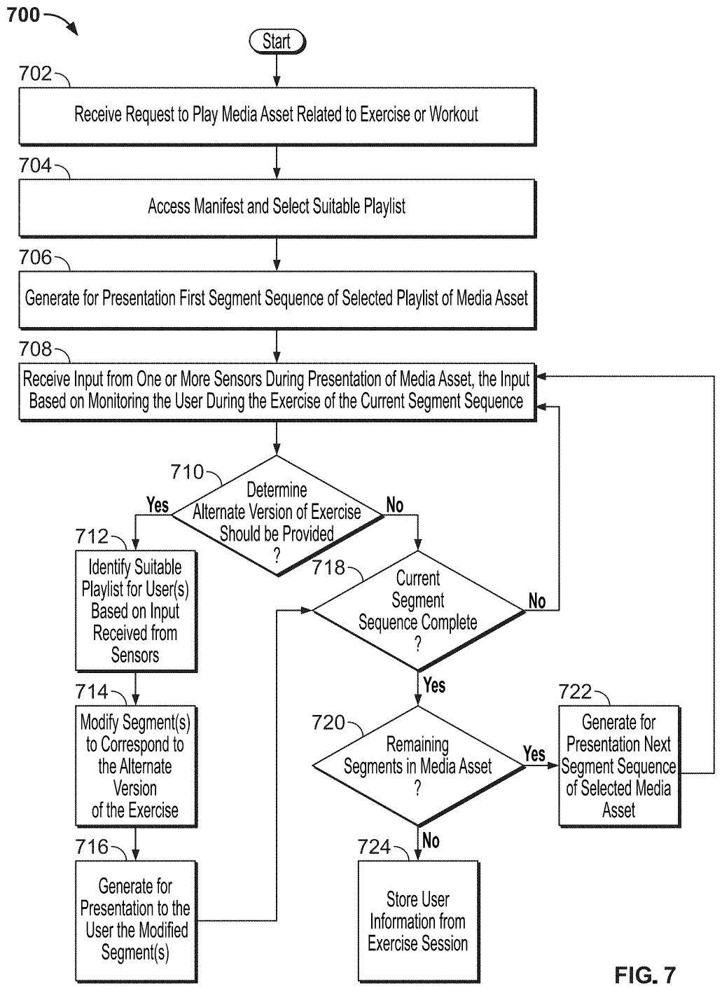

FIG. 1 shows an illustrative environment 100 in which an alternate version of an exercise depicted in a media asset may be provided to a user, in accordance with some embodiments of this disclosure. A media application (e.g., executed at least in part on a server, such as, for example, server 704 of FIG. 7, and/or user equipment 106 of FIG. 1) may receive selection of a media asset from user 102. For example, the media application may receive input (e.g., in the form of voice, touch, text, biometric, or any combination thereof) selecting option 201 of FIG. 2 to begin generating for presentation media asset 108, which may be an exercise video entitled “Core Blasting Workout.” As referred to herein, the term “media asset” should be understood to refer to an electronically consumable user asset, e.g., television programming, as well as pay-per-view programs, on-demand programs (as in video-on-demand (VOD) systems), Internet content (e.g., streaming content, downloadable content, webcasts, etc.), video clips, audio, playlists, websites, articles, electronic books, blogs, social media, applications, games, and/or any other media or multimedia, and/or combination of the above. In some embodiments, media asset 108 may be available on-demand, e.g., pre-recorded, or may be streamed or broadcast in real time. In some embodiments, media asset 108 may be broadcast or streamed on-demand or live.

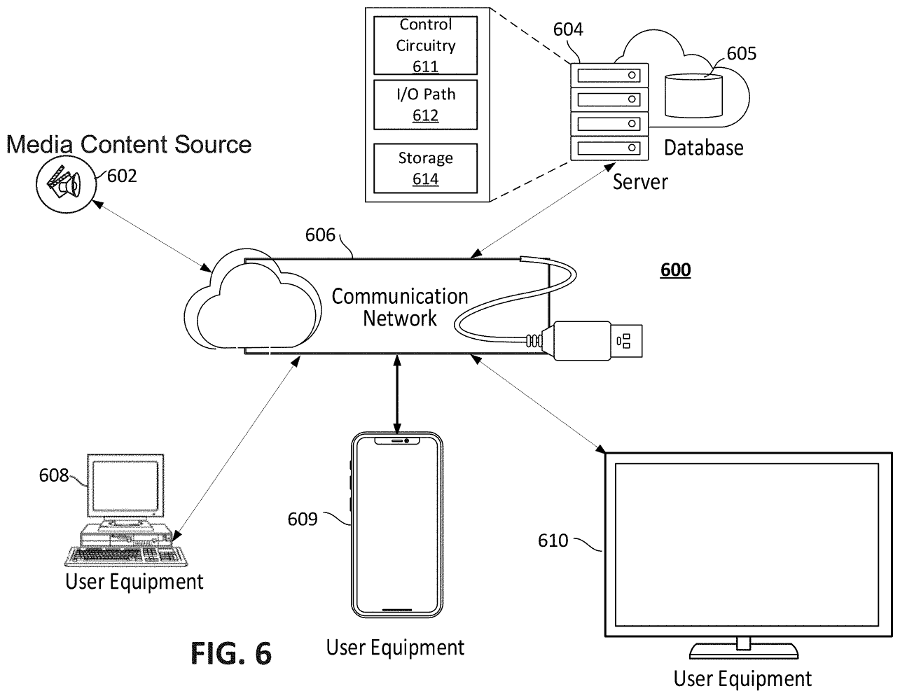

FIG. 2 shows a block diagram of an illustrative system 200 for providing an alternate version of an exercise depicted in a media asset, in accordance with some embodiments of this disclosure. Manifest 207 for media asset 108 may be stored at media server 205 (e.g., server 602 and/or server 604 of FIG. 6). As referred to herein, the term “manifest” should be understood to refer to a file and/or a data structure containing information about sequential segments (comprising sequential frames) of a media asset that is available to a client device. Such information may include, e.g., a number of segments in a playlist, bit rates of each segment, codecs associated with each segment, resolution of each segment, exercise intensity level associated with each segment, exercise experience level associated with each segment, timing of each segment, location on the network (e.g., network 606 of FIG. 6) where a segment may be retrieved, bandwidth of each segment, video tracks of each segment, audio tracks of each segment, subtitle tracks of each segment, captions of each segment, languages of each segment, other metadata associated with each segment, etc. In some embodiments, one or more segments may correspond to a particular exercise (e.g., a plank exercise of a predefined duration) from among a series of exercises depicted in a media asset.

The manifest may be employed in any of a variety of streaming protocols, e.g., media presentation description (MPD) files for Dynamic Adaptive Streaming over HTTP (MPEG-DASH), m3u8 files for HTTP Live Streaming (HLS), f4m files for HTTP Dynamic Streaming (HDS), ingest files for CMAF (Common Media Application Format), manifest files for Microsoft Smooth Streaming (MSS), etc. The manifest may be a standard manifest (e.g., an MPD file from MPEG-DASH) or may be a modified version of a standard manifest. A segment may comprise information (e.g., encoded video, audio, subtitle information, error correction bits, error detection bits, etc.) for a particular interval of a media asset, and each segment may correspond to a file specified in the manifest indicating an associated URL for retrieving the file. The segment may comprise a collection or sequence of frames (e.g., still images that together make up moving pictures of scenes of a portion of a media asset), and each segment may have a specific length (e.g., from one second to a few seconds). In the segment-based delivery of media content using the above-mentioned streaming protocols, various techniques may be employed (e.g., MPEG-2 transport stream format, MPEG-4 format such as the fragmented MPEG-4 format).

Manifest 207 may comprise various playlists that are associated with different intensity levels (e.g., “1,” which may be the most intense version of a particular exercise or round or exercises; “2,” which may be a version of the exercise or round or exercises of moderate difficulty; “3,” which may be a version of the exercise or round or exercises with less difficulty) and exercise experience levels (e.g., “Advanced,” which may be suitable for individuals who frequently exercise; “Intermediate,” which may be suitable for individuals who occasionally exercise; “Beginner,” which may be suitable for individuals who rarely exercise or are just starting to exercise). In some embodiments, the playlists of manifest 207 may comprise segments depicting a virtual instructor or workout companion 118 performing various exercises or workouts to enable user 102 to mimic the exercises or workouts during presentation of media asset 108. In some embodiments, the media application may provide the sequence of movements of the exercises as a sequence of images user 102 may follow to complete the exercises.

The playlists of manifest 207 may respectively correspond to different or alternate versions of the same or similar exercises or workouts. For example, playlist 211 of manifest 207 may be associated with intensity level 1 and an experience level of “Advanced” and may depict, e.g., from segments 16-30 (associated with a time stamp of 1:01-2:00 during presentation of media asset 108) virtual instructor 118 performing a plank exercise, which involves maintaining a push-up position for a predefined period of time or for a maximum possible time. In addition, playlist 211 may depict from segments 31-45 (e.g., associated with a time stamp of 2:01-3:00 during presentation of media asset 108) virtual instructor 118 performing a burpee exercise which involves a sequence of moves where a user jumps, squats, planks and performs a push-up, and repeats the sequence for a predefined period of time or a maximum period of time. On the other hand, playlist 213 of manifest 207 may be associated with intensity level 2 and an experience level of “Intermediate” and may depict, e.g., from segments 16-30 (associated with a time stamp of 1:01-2:00 during presentation of media asset 108) virtual instructor 118 performing a modified plank (e.g., with knees bent or knees on the floor, which is a less strenuous version of the standard plank exercise, and/or to hold the position for a shorter period of time than in the standard plank in playlist 211). In addition, playlist 213 may depict from segments 31-45 (e.g., associated with a time stamp of 2:01-3:00 during presentation of media asset 108) a “lighter” burpee exercise as compared to the standard burpee, e.g., to skip the push-up and/or to repeat the performing of the sequence of movements for less time than in playlist 211). In some embodiments, a different exercise or workout may be used in playlist 213 as a substitute for a particular exercise or workout in playlist 211, e.g., virtual instruction 118 may perform jumping jacks in playlist 213 during the corresponding segments of playlist 211 in which virtual instructor 118 performs the burpee exercise. In some embodiments, virtual instructor 118 may be the same or different across each of the playlists of manifest 207. In some embodiments, a particular workout or exercise of playlist 211 may be skipped altogether in playlist 213, e.g., in favor of a rest period.

At 209, the media application may access the manifest corresponding to selected playlist 211. In some embodiments, selection of playlist 211 may be received from user device 106 (e.g., a smart television) or user device 114 (e.g., a smartphone or tablet). Additionally or alternatively, a particular playlist may be automatically selected or recommended based on a variety of factors. For example, the media application may retrieve a user profile of user 102 associated with the media application or other exercise applications and determine a suitable playlist based on the recent workouts or frequency of workouts performed by user 102. In some embodiments, the media application may additionally or alternatively determine a suitable playlist based on information provided by the user (e.g., weight, age, height, personal goals, preferences, etc.) and/or current input received from one or more sensors (e.g., camera 104; smart watch 110 such as, for example, the Fitbit band or Apple Watch or Samsung Gear; chest strap heart rate monitor 112; a sensor associated with mobile device 114 or exercise equipment 116, pulse oximeter, etc.).

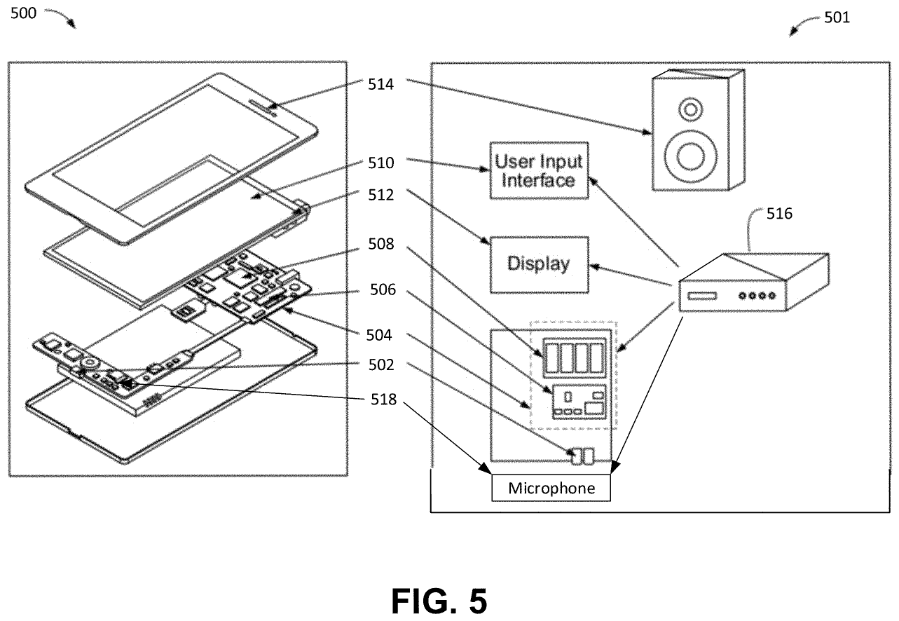

At 215, the media application, having received selection or otherwise caused selection of playlist 211 to be used in generating for presentation media asset 108, may obtain and analyze sensor data during presentation of one or more current segments being presented. In some embodiments, one or more sensors (e.g., camera 104; smart watch or Fitbit band 110; chest strap heart rate monitor 112; a sensor associated with mobile device 114 or exercise equipment 116, pulse oximeter, etc.) may be used to determine or generate sensor data during a warm-up exercise round (e.g., consecutive segments 1-15 corresponding to a time stamp of 0:00-1:00) used to assess the physical abilities of user 102 such as in the form of a fitness test. In some embodiments, each playlist of manifest 207 may have the same or similar warm-up portion (e.g., virtual instructor 118 may perform a predefined amount of push-ups, followed by a break or rest of a predefined period, followed by a predefined period of jumping rope, followed by a break or rest of a predefined period, followed by lunges, etc.), and the one or more sensors may monitor user 102 while user 102 performs the exercises being performed by virtual instructor 118. Biometric data (e.g., heart rate measurements) and/or other data (e.g., time to complete the warm-up portion of the exercise, form or posture during the warm-up exercise) of user 102 may be gathered using the sensors, including physical activity, organic function and vitals of user 102 and/or the media application may receive information from the user (e.g., calories consumed recently, exercise preferences or goals, hours of sleep the user had the prior night, etc.). In some embodiments, the media application may utilize a microphone (e.g., microphone 518 of FIG. 5) to obtain audio signals uttered by user 102 and analyze the audio signals (e.g., utilizing speech-to-text transcription) to interpret the audio signal. For example, the media application may determine that the audio signal corresponds to “This is so easy,” or “This is too hard,” or excessive screaming or sounds indicating user 102 is in pain, and take this into account when determining whether to present an alternate segment of an exercise.

In some embodiments, the media application may analyze, based on input received from the one or more sensors, and during the warm-up portion of the workout or other portions of the workout, a range of motion of user 102 (e.g., to identify improper form or posture or an asana of user 102 and/or potential muscle stiffness, spasms or cramps during an exercise; tendencies or attributes of the user such as whether the user is right- or left-handed); the agility of user 102; spatial constraints of user 102 (e.g., proximity of user 102 to walls or other structures or objects that may hinder the ability of user 102 to utilize a full range of motion for a particular exercise). The playlist of manifest 207 presented to user 102 may be dynamically updated based on the above-mentioned analysis. In some embodiments, the media application may provide feedback to the user to improve his or her posture or form (e.g., reduce the arch in the upper back region during the plank workout). In some embodiments, each sequence of segments may be tagged with various attributes pertaining to a workout or exercise session, in order to dynamically curate and/or alter the current or subsequent audiovisual track in the playlist by taking into account the user's response to the workout. For example, dance fitness segments (e.g., Zumba) may require a substantial amount of space to perform as well as a substantial amount of shoulder and leg movements, and such dance fitness segments may be tagged with attributes such as, for example, high_energy_shoulder, high_energy_leg, more_space. Based on the response of user 102 to the on going workout, the media application may extract and determine attributes of user 102 and compare the extracted attributes to the tagged dance fitness segments. In response to determining that a particular tag matches a determined attribute of the user, the media application may cause the segment associated with the particular tag to be generated for presentation in place of a current or next segment being presented. A sample matrix for a tag is shown in Table 1 below:

TABLE 1

| Tags for workout regime | | |
|---|---|---|
| Style | Zumba | |
| | Pilates | |
| | Yoga | |
| | Dance | Style of Dance-style 1, |
| | | style 2, style 3 |
| Range of | Legs restricted | Left/right/both |
| movement | Hands | Left/right/both |
| | restricted | |
| | Body | High/low/medium |
| | movements | |
| | restriction level | |
| Defect counts | Pose1 # | # Defect count |
| | Pose2 # | # Defect count |
| | Pose3 # | # Defect count |
| Spatial | Depth of room | |
| Parameters | Right/left | |
| | coordinate for | |
| | movement | |
| | Front/back | |
| | coordinate for | |
| | movement | |
| Agility Level | Range of value | |
| Style | Zumba | |
| | Pilates | |
| | Yoga | |

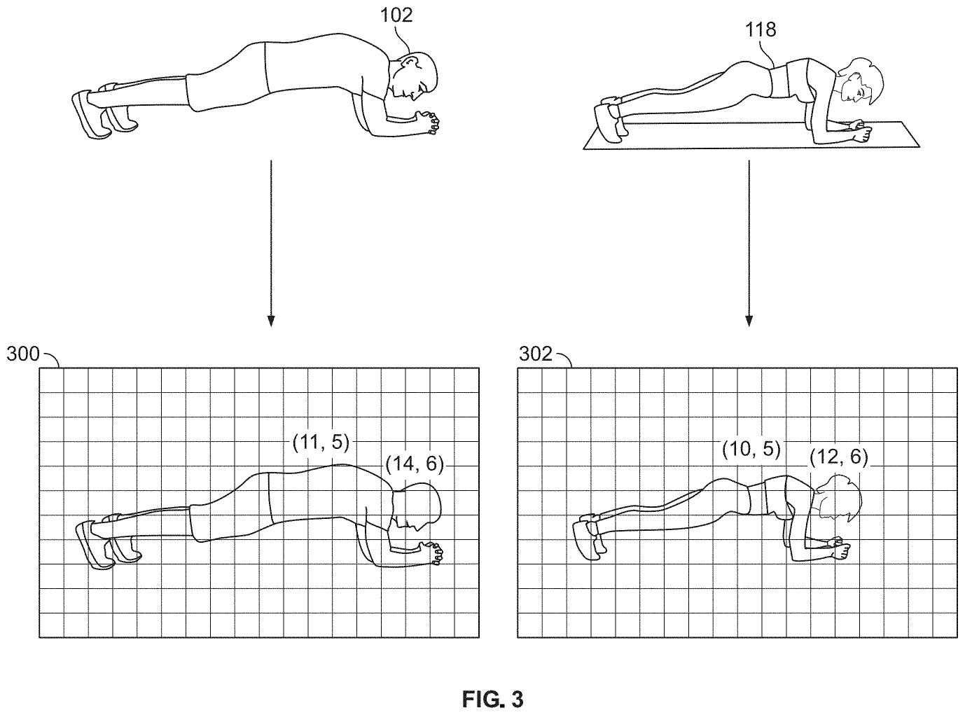

At 216, the media application determine whether an alternate version of the exercise of the current segment and/or an alternate version of the exercise of the next segment should be provided based on the sensor data obtained and analyzed at 215. For example, the media application may determine that a heart rate of user 102, determined based on smart device 110 or chest strap heart rate monitor 112, while performing the plank exercise during segments 16-30 of playlist 211, exceeds a predefined threshold. The threshold may be set based on the characteristics of user 102 and the attributes of the workout. Additionally or alternatively, the media application may determine, based on images captured by camera 104, that user 102 is unable to hold the push-up position of the plank (e.g., continues to put his knees on the ground during the exercise) and/or may analyze the form or posture of user 102 during the plank exercise, as discussed in more detail in connection with FIG. 3. Based on such analysis, the media application may, at 216, determine that user 102 is performing the current exercise sufficiently well, e.g., with his biometrics being at an acceptable level, and thus, at 219, continue presenting segments of playlist 211. On the other hand, the media application may, at 221, access manifest 207 to identify playlist 213, in response to determining that an alternate exercise should be presented to the user. For example, the media application may retrieve or fetch video and/or audio tracks associated with segments 31-45 of playlist 213 of the lighter burpee (e.g., less strenuous than segments 31-45 of playlist 211), based on the form of user 102 during the plank exercise of playlist 211 being suboptimal or the heart rate of user 102 during the plank exercise exceeding the predefined threshold. In this way, exercises may be personalized to user 102 in real time based on his current physical capabilities.

In some embodiments, the media application may alter a current segment of the exercise being presented (e.g., switch from depicting virtual instructor 118 performing the plank of playlist 211 to virtual instructor 118 performing the modified plank of playlist 213) in addition to, or alternative to, adjusting the exercise of one or more of the next segments based on the projected state of the user (e.g., the media application may infer that, if the user is unable to perform the plank at an acceptable level, he or she is unlikely to be able to perform the standard burpee exercise at an acceptable level). In some embodiments, the media application may determine based on the analyzed sensor data that user 102 should be provided with an alternate, more difficult version of a workout. For example, the media application may determine that biometric data of the user indicates he or she is not over-exerting himself or herself, that user 102 completed the exercise in the allotted time or less than the allotted time, that user 102 is complaining the exercise is too easy, and/or that the form of user 102 is optimal. In such an instance, the media application may retrieve segments corresponding to a more difficult version of a current or future exercise (e.g., retrieve segments for “Advanced” instead of “Intermediate” exercises being performed by user 102). For example, a particular playlist may be selected that specifies for such a user an “extended plank” (e.g., depicting virtual instructor 118 extending her body further forward) which may add difficulty to the standard plank exercise of playlist 211. In some embodiments, in the context of a live exercise video, a particular segment may be skipped based on the input from the one or more sensors, or the media application may provide a warning notification to the user regarding the difficulty of the next exercise coupled with the current status of the user.

At 223, the media application may generate for presentation the modified segments (e.g., remaining segments of modified plank of playlist 213 and/or segments of the lighter burpee of playlist 213 in place of the corresponding segments of playlist 211). In some embodiments, the media application may provide user 102 an option that is selectable to switch back to the prior version of the workout (e.g., to switch back to playlist 211 from playlist 213). The media application may reevaluate the performance of user 102 and biometric information of user 102 during each group of segments to determine whether alternate versions of an exercise should be presented. In some embodiments, the media application may compare a biometric response of user 102 to those of other users performing a similar exercise and/or having similar traits to user 102, and determine whether an alternate version of an exercise should be presented, based on whether the biometric response of user 102 falls within an particular range of biometric responses computed based on the biometric responses of the plurality of other users.

As referred to herein, the modifying of the at least one segment performed at 223 of FIG. 2 may be understood as replacing a particular version of an exercise (e.g., a plank) that is scheduled to be depicted in a media asset being consumed by a user with a less intense (e.g., a modified plank which may be less strenuous than a standard plank) or more intense (e.g., an extended plank which may be more strenuous than a standard plank) version of the same workout or with a less intense or more intense version of a similar workout (e.g., a different exercise of varying intensity targeting similar muscle groups as the scheduled exercise, such as if a particular user profile indicates the user is suffering from an injury that suggests he or she should not perform the scheduled workout or variations thereof).

In some embodiments, the media application may take into account other information in determining whether to present an additional workout. For example, the media application may receive information indicative of nutrition (e.g., calories, proteins, carbohydrates, water, etc.) recently consumed by user 102 (e.g., during the past 24 hours, or during the past 12 hours) and use this information as a factor in determining an intensity level of a workout to be retrieved. For example, if the media application receives information indicating user 102 may be dehydrated or has not consumed much food recently, this may weigh in favor of a less intense workout. As another example, the media application may receive information, e.g., entered by user 102 into his media application profile, regarding an existing injury of user 102. The media application may query a database (e.g., database 605 of FIG. 6) storing anonymized workout data from a plurality of users, which may comprise a table of exercises and movements and correlated physical injuries, and recommend an alternate exercise based on the table (e.g., to avoid certain exercises that may aggravate the reported injury of user 102 in favor of exercises that are less likely to aggravate the reported injury of user 102). For example, the plurality of users may report via the media application in real time an injury suffered during a particular workout or exercise being performed in order to link or correlate the exercise with the injury.

In some embodiments, data captured by one or more sensors (e.g., camera 104) may be used to extract 3D skeletal information of the plurality of users (e.g., based on the coordinates of the body parts of the user as shown in FIG. 3 as well as detected depth information), and recorded video of the skeletal information may be analyzed to determine if an injury was a result of improper form or posture of the user during the exercise. Such skeletal information may be utilized instead of actual video of users to preserve anonymity. Skeletal data recognition algorithms are discussed in more detail in connection with Shotton et al., “Real-Time Human Pose Recognition in Parts from Single Depth Images,” The 24th IEEE Conference on Computer Vision and Pattern Recognition, CVPR 2011, Colorado Springs, Colo., USA, 20-25 Jun. 2011, which is hereby incorporated by reference in its entirety. In some embodiments, captured data from multiple users in a workout session (e.g., a virtual group workout) or other distinct sessions may be collected and analyzed to determine consistencies in form among multiple users, analyze movements that potentially resulted in injuries, and recommend alterations in form or posture to a user in a certain group (e.g., beginner) based on the form or posture of users in another group (e.g., advanced athletes) and/or data from users that progressed from a beginner level to an intermediate level and an advanced level in a short period of time (e.g., less than a predefined threshold period of time).

In some embodiments, exercise equipment 116 (e.g., dumbbells, barbells, exercise bike, treadmill, or any other suitable exercise machine or equipment) may be used by user 102 based on instructions from virtual instructor 118. In some embodiments, exercise equipment 116 may be “smart” equipment comprising one or more sensors (e.g., an accelerometer) to count repetitions of an exercise (e.g., a bicep curls with dumbbells) as well as how much weight is being lifted and a time required to perform the repetitions. In some embodiments, this information may be wirelessly communicated (e.g., via Wi-Fi or Bluetooth) to the media application. Exercise equipment 116 may include one or more mechanisms (e.g., dials or rotation mechanism) to add or remove plates of weight from the equipment, to enable performing exercises with varying amounts of weight. In some embodiments, the media application may recommend a weight amount for exercise equipment 116 for a particular exercise or workout based on the biometric or other collected information of user 102 and transmit such recommendation to exercise equipment 116. Exercise equipment 116 may be configured to automatically adjust the mechanism to vary the amount of weight for the exercise based on the recommendation received from the media application, e.g., signals received from the media application may be used to control a mechanical motor (which may not be part of the portion of equipment 116 lifted by user 102) to adjust the weight or resistance of exercise equipment 116.

FIG. 3 shows a block diagram of an illustrative technique for identifying posture or form of a user during an exercise, in accordance with some embodiments of this disclosure. Posture information 300 corresponds to the posture of user 102 of FIG. 1 during a plank exercise, and posture information 302 corresponds to the posture of virtual instructor 118 of FIG. 1 during the plank exercise. Using image-processing methods, e.g., object recognition, facial recognition, edge detection, or any other suitable image processing method, the media application identifies portions of the body and appearance of by user 102 and the media application determines the position of each identified portion. In the example of FIG. 3, a Cartesian coordinate plane is used to identify the position of each identified portion of the body and appearance of user 102, with the position recorded as (X,Y) coordinates on the plane. For example, the upper region of the back of user 102 may span from (11, 5) to (14, 6), whereas the upper region of the back of user 118 may span from (10, 5) to (12, 6). In some embodiments, the coordinates may include a coordinate in the Z-axis, to identify the position of each identified portion of the body and appearance of user 102 in 3D space, based on images captured using 3D sensors and any other suitable depth-sensing technology. The media application may compare the coordinates for certain regions of the body and appearance of user 102 to the body and appearance of virtual instructor 118 to determine whether the form of user 102 is proper (e.g., based on the assumption that the form of virtual instructor 118 is proper). In some embodiments, to account for the differences in body type of user 102 and virtual instructor 118, the media application may normalize the coordinates of user 102 and virtual instructor 118 into a particular range (e.g., in between 0 and 1) to enable comparison of normalized values. In some embodiments, the media application may identify attributes of user 102 (e.g., height, weight) based on signals received from the one or more sensors or other information, and reference a database to identify users having similar attributes to user 102 and having been classified as having proper form during a similar exercise. Based on the comparison, the media application may determine whether the form or posture of user 102 is proper and determine whether alternate versions of an exercise and/or feedback should be provided to user 102.

In some embodiments, an exercise media asset may correspond to a yoga or a Pilates class. The yoga exercise media asset may comprise one or more segments depicting a virtual instructor standing on only one foot for a predefined period of time, where the goal of the exercise is to maintain one's balance in the position and minimize movement. The media application may monitor the performance of the user to which the media asset is being provided during this exercise. For example, one or more snapshots (e.g., a still image and/or a video) may be captured by camera 104 (which may be a stereo camera system capable of capturing images from a plurality of angles to generate 3D image data, or a single camera configured to capture images from multiple locations to generate 3D image data) to capture depth information of the user, to determine the current and/or projected state of the user. If the user is not performing the exercise well (e.g., the media application determines moderate motion based on the captured images indicating that the user is losing his or her balance and struggling to maintain the form of standing on one foot only), the media application may determine that the user is likely a beginner and is unlikely to tolerate equally intense or more intense exercises in future segments that require balancing oneself, and may modify the playlist of the media asset accordingly.

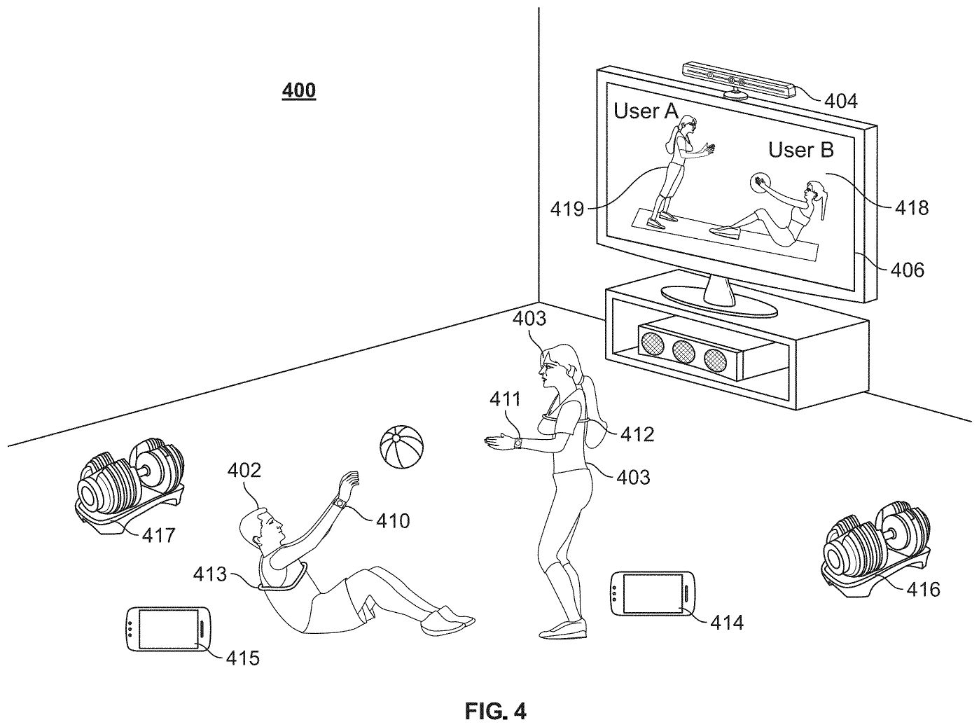

FIG. 4 shows an illustrative environment in which an alternate version of an exercise depicted in a media asset may be provided to multiple users in a joint exercise, in accordance with some embodiments of this disclosure. In environment 400, each of users 402 and 403 may be participating in an exercise associated with media asset 408. The media application may identify one or more of user 402 and user 403 using any suitable technique. For example, the media application may receive an indication that each of user 402 and 403 is participating in the exercise via a log-in screen. In some embodiments, the media application may employ facial recognition to identify user 402 and 403. For example, the media application may store a profile for each of user 402 and user 403 respectively associated with images of users 402 and 403. Camera 404 may capture images of users 402 and 403 and analyze the faces of the users in the captured images to identify the users. For example, the media application may utilize any suitable facial recognition algorithms and/or image processing techniques to identify or extract various features (e.g., distance between eyes, distance from chin to forehead, shape of jawline, depth of eye sockets, height of checkbones, overall arrangement of facial features, size and shape of facial features, etc.) of the face of the users in the image, and compare the identified facial features to the images of the users stored in connection with their respective profiles to identify the users.

In some embodiments, the media application may monitor the performance and/or biometric information of users 402 and 403 (e.g., during a warm-up portion of exercise media asset 408) based on input received from one or more sensors (e.g., camera 404; wearable devices 410, 411; chest strap heart rate 413; mobile devices 414, 415). Based on the monitored performance and/or other information received from the users (e.g., nutrition information or other preferences), the media application may determine that one of the users (e.g., user 402) is better equipped than another user (e.g., user 403) to handle a more strenuous exercise at the current time. In this instance, the media application may recommend a joint exercise for each of users 402 and 403 to participate in, where the media application may recommend that user 402 (e.g., “User B”), determined to be better equipped to handle a more intense workout, perform the more intense portion of the joint workout (e.g., performing sit-ups and tossing a medicine ball to user 403). In addition, the media application may recommend that user 403, determined to be at the current time better equipped for a less intense workout, may perform the less strenuous portion of the joint exercise of catching the medicine ball from user 402 and tossing the medicine ball back to user 402 at a suitable time). In some embodiments, the media application may recommend and/or automatically cause respective exercise equipment 416, 417 to adjust respective weight or resistance thereof based on the monitored performance and/or biometric information of user 402 and user 403. For example, if the media application determines that user 403 is likely to be capable of lifting a larger amount of weight for a particular exercise than user 402, the media application may recommend or cause exercise equipment 417 to be adjusted to a higher weight or resistance than exercise equipment.

In some embodiments, as the joint exercise media asset transitions from segment to segment, the determination of the user that is better equipped to handle a more intense portion of a joint workout may change (e.g., the input from the sensors may indicate that user 402 has become tired as a result of performing the intense workout depicted in FIG. 4). Thus, the media application may dynamically determine (e.g., for each new group of segments corresponding to a new exercise) that a different user (e.g., user 403 or “User A”) should perform the more intense workout portion of the next sequence of segments. In some embodiments, a split screen may be provided to enable user 402 and 403 to perform different workouts determined to be suitable for the respective users current performance state and/or biometric information (e.g., if a joint workout is indicated as not desirable).

FIGS. 5-6 describe exemplary devices, systems, servers, and related hardware for providing an alternate version of an exercise depicted in a media asset, in accordance with some embodiments of the present disclosure. FIG. 5 shows generalized embodiments of illustrative user equipment devices 500 and 501, which may correspond to user equipment devices 106, 114 of FIG. 1; 214 of FIGS. 2; 406 and 414 of FIG. 4. For example, user equipment device 500 may be a smartphone device. In another example, user equipment system 501 may be a user television equipment system. User television equipment system 501 may include set-top box 516. Set-top box 516 may be communicatively connected to microphone 518, speaker 514, and display 512. In some embodiments, microphone 518 may receive voice commands and/or detect audio reactions to particular exercises in connection with the media application. In some embodiments, display 512 may be a television display or a computer display. In some embodiments, set-top box 516 may be communicatively connected to user input interface 510. In some embodiments, user input interface 510 may be a remote control device. Set-top box 516 may include one or more circuit boards. In some embodiments, the circuit boards may include processing circuitry, control circuitry, and storage (e.g., RAM, ROM, hard disk, removable disk, etc.). In some embodiments, the circuit boards may include an input/output path. More specific implementations of user equipment devices are discussed below in connection with FIG. 6. Each one of user equipment device 500 and user equipment system 501 may receive content and data via input/output (I/O) path 502. I/O path 502 may provide content (e.g., broadcast programming, on-demand programming, Internet content, content available over a local area network (LAN) or wide area network (WAN), and/or other content) and data to control circuitry 504, which includes processing circuitry 506 and storage 508. Control circuitry 504 may be used to send and receive commands, requests, and other suitable data using I/O path 502, which may comprise I/O circuitry. I/O path 502 may connect control circuitry 504 (and specifically processing circuitry 506) to one or more communications paths (described below). I/O functions may be provided by one or more of these communications paths, but are shown as a single path in FIG. 5 to avoid overcomplicating the drawing.

Control circuitry 504 may be based on any suitable processing circuitry such as processing circuitry 506. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, control circuitry 504 executes instructions for the media application stored in memory (e.g., storage 508). Specifically, control circuitry 504 may be instructed by the media application to perform the functions discussed above and below. In some implementations, any action performed by control circuitry 504 may be based on instructions received from the media application.

In client/server-based embodiments, control circuitry 504 may include communications circuitry suitable for communicating with a media application server or other networks or servers. The instructions for carrying out the above mentioned functionality may be stored on a server (which is described in more detail in connection with FIG. 6). Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communication networks or paths (which is described in more detail in connection with FIG. 6). In addition, communications circuitry may include circuitry that enables peer-to-peer communication of user equipment devices, or communication of user equipment devices in locations remote from each other (described in more detail below).

Memory may be an electronic storage device provided as storage 508 that is part of control circuitry 504. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage 508 may be used to store various types of content described herein as well as media application data described above. Nonvolatile memory may also be used (e.g., to launch a boot-up routine and other instructions). Cloud-based storage, described in relation to FIG. 4, may be used to supplement storage 508 or instead of storage 508.

Control circuitry 504 may include video generating circuitry and tuning circuitry, such as one or more analog tuners, one or more MPEG-2 decoders or other digital decoding circuitry, high-definition tuners, or any other suitable tuning or video circuits or combinations of such circuits. Encoding circuitry (e.g., for converting over-the-air, analog, or digital signals to MPEG signals for storage) may also be provided. Control circuitry 504 may also include scaler circuitry for upconverting and downconverting content into the preferred output format of user equipment 500. Control circuitry 504 may also include digital-to-analog converter circuitry and analog-to-digital converter circuitry for converting between digital and analog signals. The tuning and encoding circuitry may be used by user equipment device 500, 501 to receive and to display, to play, or to record content. The tuning and encoding circuitry may also be used to receive guidance data. The circuitry described herein, including for example, the tuning, video generating, encoding, decoding, encrypting, decrypting, scaler, and analog/digital circuitry, may be implemented using software running on one or more general purpose or specialized processors. Multiple tuners may be provided to handle simultaneous tuning functions (e.g., watch and record functions, picture-in-picture (PIP) functions, multiple-tuner recording, etc.). If storage 508 is provided as a separate device from user equipment device 500, the tuning and encoding circuitry (including multiple tuners) may be associated with storage 508.

Control circuitry 504 may receive instruction from a user by way of user input interface 510. User input interface 510 may be any suitable user interface, such as a remote control, mouse, trackball, keypad, keyboard, touch screen, touchpad, stylus input, joystick, voice recognition interface, or other user input interfaces. Display 512 may be provided as a stand-alone device or integrated with other elements of each one of user equipment device 500 and user equipment system 501. For example, display 512 may be a touchscreen or touch-sensitive display. In such circumstances, user input interface 410 may be integrated with or combined with display 512. Display 512 may be one or more of a monitor, a television, a display for a mobile device, or any other type of display. A video card or graphics card may generate the output to display 512. The video card may be any processing circuitry described above in relation to control circuitry 504. The video card may be integrated with the control circuitry 504. Speakers 514 may be provided as integrated with other elements of each one of user equipment device 500 and user equipment system 501 or may be stand-alone units. The audio component of videos and other content displayed on display 512 may be played through the speakers 514. In some embodiments, the audio may be distributed to a receiver (not shown), which processes and outputs the audio via speakers 514.

The media application may be implemented using any suitable architecture. For example, it may be a stand-alone application wholly-implemented on each one of user equipment device 500 and user equipment system 501. In such an approach, instructions of the application are stored locally (e.g., in storage 508), and data for use by the application is downloaded on a periodic basis (e.g., from an out-of-band feed, from an Internet resource, or using another suitable approach). Control circuitry 504 may retrieve instructions of the application from storage 508 and process the instructions to rearrange the segments as discussed. Based on the processed instructions, control circuitry 504 may determine what action to perform when input is received from user input interface 510. For example, movement of a cursor on a display up/down may be indicated by the processed instructions when user input interface 510 indicates that an up/down button was selected.

In some embodiments, the media application is a client/server-based application. Data for use by a thick or thin client implemented on each one of user equipment device 500 and user equipment system 501 is retrieved on-demand by issuing requests to a server remote to each one of user equipment device 500 and user equipment system 501. In one example of a client/server-based guidance application, control circuitry 504 runs a web browser that interprets web pages provided by a remote server. For example, the remote server may store the instructions for the application in a storage device. The remote server may process the stored instructions using circuitry (e.g., control circuitry 504) to perform the operations discussed in connection with FIGS. 1-3.

In some embodiments, the media application may be downloaded and interpreted or otherwise run by an interpreter or virtual machine (e.g., run by control circuitry 504). In some embodiments, the media application may be encoded in the ETV Binary Interchange Format (EBIF), received by the control circuitry 504 as part of a suitable feed, and interpreted by a user agent running on control circuitry 504. For example, the media application may be an EBIF application. In some embodiments, the media application may be defined by a series of JAVA-based files that are received and run by a local virtual machine or other suitable middleware executed by control circuitry 504. In some of such embodiments (e.g., those employing MPEG-2 or other digital media encoding schemes), the media application may be, for example, encoded and transmitted in an MPEG-2 object carousel with the MPEG audio and video packets of a program.

FIG. 6 is a diagram of an illustrative media system for providing an alternate version of an exercise depicted in a media asset, in accordance with some embodiments of this disclosure. User equipment devices 608, 609, 610 (e.g., user equipment device 106, 114 of FIG. 1, user equipment device 214 of FIG. 2, user equipment device 406, 414, 415 of FIG. 2) may be coupled to communication network 606. Communication network 606 may be one or more networks including the Internet, a mobile phone network, mobile voice or data network (e.g., a 5G, 4G, or LTE network), cable network, public switched telephone network, or other types of communication network or combinations of communication networks. Paths (e.g., depicted as arrows connecting the respective devices to the communication network 606) may separately or together include one or more communications paths, such as a satellite path, a fiber-optic path, a cable path, a path that supports Internet communications (e.g., IPTV), free-space connections (e.g., for broadcast or other wireless signals), or any other suitable wired or wireless communications path or combination of such paths. Communications with the client devices may be provided by one or more of these communications paths but are shown as a single path in FIG. 6 to avoid overcomplicating the drawing.

Although communications paths are not drawn between user equipment devices, these devices may communicate directly with each other via communications paths as well as other short-range, point-to-point communications paths, such as USB cables, IEEE 1394 cables, wireless paths (e.g., Bluetooth, infrared, IEEE 702-11x, etc.), or other short-range communication via wired or wireless paths. The user equipment devices may also communicate with each other directly through an indirect path via communication network 606.

System 600 includes a media content source 602 and a server 604, which may comprise or be associated with database 605 (e.g., user information database storing exercise information of a plurality of users and profile information regarding the users). Communications with media content source 602 and server 604 may be exchanged over one or more communications paths but are shown as a single path in FIG. 6 to avoid overcomplicating the drawing. In addition, there may be more than one of each of media content source 602 and server 604, but only one of each is shown in FIG. 6 to avoid overcomplicating the drawing. If desired, media content source 602 and server 604 may be integrated as one source device.

In some embodiments, server 604 may include control circuitry 611 and a storage 614 (e.g., RAM, ROM, Hard Disk, Removable Disk, etc.). Storage 614 may store one or more databases (e.g., user information database storing exercise information of a plurality of users and profile information regarding the users). Server 604 may also include an input/output path 612. I/O path 612 may provide device information, or other data, over a local area network (LAN) or wide area network (WAN), and/or other content and data to the control circuitry 611, which includes processing circuitry, and storage 614. The control circuitry 611 may be used to send and receive commands, requests, and other suitable data using I/O path 612, which may comprise I/O circuitry. I/O path 612 may connect control circuitry 604 (and specifically processing circuitry) to one or more communications paths.

Control circuitry 611 may be based on any suitable processing circuitry such as one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, control circuitry 611 may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, the control circuitry 611 executes instructions for an emulation system application stored in memory (e.g., the storage 614). Memory may be an electronic storage device provided as storage 614 that is part of control circuitry 611.

Server 604 may retrieve guidance data from media content source 602, process the data as will be described in detail below, and forward the data to user equipment devices 608, 609, 610. Media content source 602 may include one or more types of content distribution equipment including a television distribution facility, cable system headend, satellite distribution facility, programming sources (e.g., television broadcasters, such as NBC, ABC, HBO, etc.), exercise programming sources (e.g., Peloton, Samsung Health, Amazon Prime Fitness, Apple Fitness, NordicTrack, Lululemon Mirror, etc.), intermediate distribution facilities and/or servers, Internet providers, on-demand media servers, and other content providers. NBC is a trademark owned by the National Broadcasting Company, Inc., ABC is a trademark owned by the American Broadcasting Company, Inc., and HBO is a trademark owned by the Home Box Office, Inc. Media content source 602 may be the originator of content (e.g., a television broadcaster, a Webcast provider, etc.) or may not be the originator of content (e.g., an on-demand content provider, an Internet provider of content of broadcast programs for downloading, etc.). Media content source 602 may include cable sources, satellite providers, on-demand providers, Internet providers, over-the-top content providers, or other providers of content. Media content source 602 may also include a remote media server used to store different types of content (including video content selected by a user), in a location remote from any of the client devices. Media content source 602 may provide exercise videos associated with a plurality of segments and alternate versions of exercises as described above.

Client devices may operate in a cloud computing environment to access cloud services. In a cloud computing environment, various types of computing services for content sharing, storage or distribution (e.g., video sharing sites or social networking sites) are provided by a collection of network-accessible computing and storage resources, referred to as “the cloud.” For example, the cloud can include a collection of server computing devices (such as, e.g., server 604), which may be located centrally or at distributed locations, that provide cloud-based services to various types of users and devices connected via a network such as the Internet via communication network 606. In such embodiments, user equipment devices may operate in a peer-to-peer manner without communicating with a central server.

FIG. 7 is a flowchart of a detailed illustrative process for providing an alternate version of an exercise depicted in a media asset, in accordance with some embodiments of this disclosure. In various embodiments, the individual steps of process 700 may be implemented by one or more components of the devices and systems of FIGS. 1-6. Although the present disclosure may describe certain steps of process 700 (and of other processes described herein) as being implemented by certain components of the devices and systems of FIGS. 1-6, this is for purposes of illustration only, and it should be understood that other components of the devices and systems of FIGS. 1-6 may implement those steps instead. For example, the steps of process 700 may be executed at device 609 and/or server 604 of FIG. 6 to perform the steps of process 700.

At 702, control circuitry (e.g., control circuitry 504 of FIG. 5 and/or control circuitry 611 of FIG. 6) may be configured to receive a request to play a media asset related to a physical exercise or workout (e.g., media asset 108 of FIG. 1, based on receiving selection of media asset identifier 201 of “Core Blasting Workout” via user device 214 of FIG. 2). In some embodiments, the control circuitry may receive log-in or other identifying information of the user prior to providing access to the media asset, e.g., via voice, touch, text, biometric input, or any combination thereof).

At 704, control circuitry (e.g., control circuitry 504 of FIG. 5 and/or control circuitry 611 of FIG. 6) may access a manifest (e.g., manifest 207 of FIG. 2), which may be stored at a media server (e.g., media server 205 of FIG. 2). Manifest 207 may identify the network address of various playlists (e.g., playlists 211, 213) and segments thereof that may be associated with similar exercises of various intensity and experience levels to accommodate users of varying physical capabilities. In some embodiments, the control circuitry may receive a selection of a particular playlist (e.g., “Advanced,” “Intermediate,” or “Beginner”). In some embodiments, the control circuitry may receive input from one or more sensors (e.g., camera 104; smart watch 110 such as, for example, a Fitbit band or Apple Watch or Samsung Gear; chest strap heart rate monitor 112; a sensor associated with mobile device 114 or exercise equipment 116 of FIG. 1, etc.) to determine a suitable playlist based on the physical condition or appearance of the user. In some embodiments, the control circuitry may additionally or alternatively take into consideration a workout history associated with a profile of the user (e.g., user 102 of FIG. 1) to identify which playlist to select.

At 706, control circuitry (e.g., control circuitry 504 of FIG. 5 and/or control circuitry 611 of FIG. 6) may generate for presentation a first segment sequence of the selected playlist (e.g., playlist 211 of FIG. 2) of the media asset (e.g., media asset 108 of FIG. 1). For example, the first segment sequence may correspond to segments 1-15 associated with a time stamp of 0:00-1:00 and may enable user equipment (e.g., user device 214 and/or user equipment 106) to depict audio and visual elements of a warm-up exercise to the user.

At 708, control circuitry (e.g., control circuitry 504 of FIG. 5 and/or control circuitry 611 of FIG. 6) may receive input from one or more sensors e.g., camera 104; smart watch 110 such as, for example, a Fitbit band or Apple Watch or Samsung Gear; chest strap heart rate monitor 112; a sensor associated with mobile device 114 or exercise equipment 116 of FIG. 1, etc.) during presentation of the media asset (e.g., media asset 108 of FIG. 1). For example, the control circuitry may analyze the form of the user (e.g., user 102 of FIG. 1) during the warm-up portion of the exercise, how quickly the user completes certain workouts, a heart rate of the user during the workout, etc. In some embodiments, the control circuitry may determine the spatial constraints of the user, e.g., whether the workout space the user is in is large enough to accommodate a full range of motion of various exercises, using 3D depth sensing techniques (e.g., using camera 104 of FIG. 1).

At 710, control circuitry (e.g., control circuitry 504 of FIG. 5 and/or control circuitry 611 of FIG. 6) may determine, based on the received input from the one or more sensors, whether an alternate version of a particular workout (e.g., the current exercise of the current sequence of segments and/or the next exercise of the next sequence of segments) should be provided. For example, the control circuitry may determine that the user, (e.g., user 102 of FIG. 1), is performing the warm-up exercise of playlist 211 adequately, and his or her biometric data is within an acceptable range, and thus he should receive the subsequent sequence of segments for the plank exercise of playlist 211. On the other hand, the control circuitry may determine that the user is struggling with the warm-up exercises and that the biometric data of the user falls outside an acceptable range (e.g., which may be determined based on an analysis of anonymized data of a plurality of users ,heart rates during the same or a similar exercise). In this instance, the control circuitry may determine that a lighter-intensity sequence of segments should be provided immediately (e.g., for the rest of the current sequence of segments) and/or for the next sequence of segments (e.g., segments corresponding to the “Modified Plank” workout of playlist 213, which may be a lighter-intensity version of the “Plank” workout of playlist 211. In some embodiments, the control circuitry may determine that the user is excelling at a particular sequence of segments (e.g., is using proper form and completing the required movements quickly) and thus may suggest a more intense exercise than is scheduled in the current playlist for the next sequence of segments.

At 712, control circuitry (e.g., control circuitry 504 of FIG. 5 and/or control circuitry 611 of FIG. 6), in response to determining that an alternate version of an exercise should be provided, may identify a suitable playlist (e.g., “Intermediate” playlist 213 of FIG. 2, if the user is struggling with the “Advanced” playlist), based on the input received from the one or more sensors. On the other hand, if the control circuitry determines that the user is struggling significantly (e.g., the user's form is very poor and/or he or she appears to be unable to complete the exercises of the current playlist and/or the biometric data of the user is far outside a desired range), the playlist of “Beginner” may be determined as suitable. In some embodiments, the control circuitry may determine that multiple users (e.g., users 402 and 403) are participating in, or desire to participate in, an exercise, and may determine whether such multiple users are interested in a joint workout (e.g., based on prompting the users to reply to a query of whether they would prefer a joint workout rather than each individually performing the workout of the virtual instructor of the media asset). In some embodiments, a joint workout may be selected by default upon detecting multiple users (e.g., via facial recognition or other received input). The control circuitry may retrieve a playlist for a suitable joint workout and generate for display multiple virtual instructors 418, 419. In some embodiments, the control circuitry may determine (e.g., based on sensor input) which of the users is better equipped to handle a more intense workout, and select a joint workout where the user identified as better equipped to handle the intense workout (e.g., user 402 of FIG. 4) is assigned the more strenuous portion of a workout as compared to the other user (e.g., user 403 of FIG. 4).

At 714, control circuitry (e.g., control circuitry 504 of FIG. 5 and/or control circuitry 611 of FIG. 6), may modify the segment(s), e.g., segments 16-30 of the “Plank” exercise of playlist 211, to correspond to the alternate version of the exercise, e.g., segments 16-30 of the “Modified Plank” exercise of playlist 213.

At 716, the control circuitry may generate for presentation the modified segment(s) to the user, e.g., the “Modified Plank” exercise of playlist 213, and continue to monitor the performance of the user via the one or more sensors during the modified segment(s).

At 718, control circuitry (e.g., control circuitry 504 of FIG. 5 and/or control circuitry 611 of FIG. 6), having either determined that the alternate version of the exercise should not be provided, or having already provided the alternate version of the exercise, may determine whether a current segment sequence (e.g., segments 16-30 of playlist 213 of FIG. 2) is complete. For example, the control circuitry may compare the current presentation position of the media asset (e.g., media asset 108) to the time stamp information of segments 16-30. In response to determining the current segment is not complete, processing may continue to 708 to continue monitoring the performance of the user (e.g., user 102 of FIG. 1) during the current exercise. In response to determining the current segment is complete, processing may proceed to 720.

At 720, the control circuitry may determine whether any segments remain in the media asset, e.g., any segment sequences corresponding to an additional exercise of the selected media asset (e.g., media asset 108 of FIG. 1). If it is determined that no segments corresponding to an exercise remain, processing may proceed to 724, where user data (e.g., biometric data, form or posture information during the exercises, etc.) may be stored in connection with a profile of the user (e.g., user 102 of FIG. 1). If segments of the exercise remain, processing may proceed to 722.

At 722, control circuitry (e.g., control circuitry 504 of FIG. 5 and/or control circuitry 611 of FIG. 6) may generate for presentation the next segment sequence of the selected media asset (e.g., media asset 108 of FIG. 1). For example, the control circuitry may identify “Lighter Burpee” corresponding to segments 31-45 of playlist 213 as the next sequence of segments in the exercise routine provided by the media asset, and processing may proceed to 708 to analyze the performance of the user (e.g., user 102 of FIG. 1) during such exercise portion. In some embodiments, the next segment sequence may be selected from a different playlist (e.g., playlist 211 or playlist 217) based on user selection of a particular playlist or based on the performance of the user in modified segments(s) presented at 716.

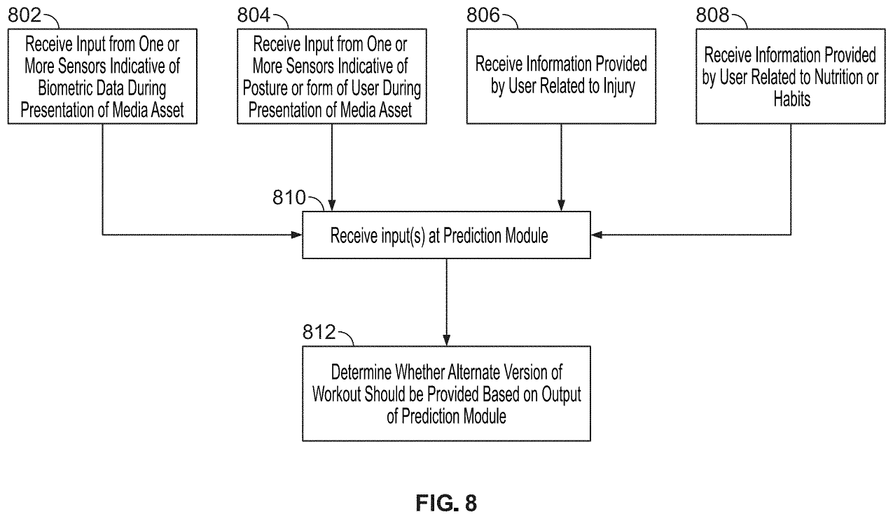

FIG. 8 is a flowchart of a detailed illustrative process for determining whether to provide an alternate version of an exercise depicted in a media asset, in accordance with some embodiments of this disclosure. In various embodiments, the individual steps of process 800 may be implemented by one or more components of the devices and systems of FIGS. 1-6. Although the present disclosure may describe certain steps of process 800 (and of other processes described herein) as being implemented by certain components of the devices and systems of FIGS. 1-6, this is for purposes of illustration only, and it should be understood that other components of the devices and systems of FIGS. 1-6 may implement those steps instead. For example, the steps of process 700 may be executed at device 609 and/or server 604 of FIG. 6 to perform the steps of process 800.

At 802, control circuitry (e.g., control circuitry 504 of FIG. 5 and/or control circuitry 611 of FIG. 6) may receive input from one or more sensors (e.g., camera 104; smart watch 110 such as, for example, a Fitbit band or Apple Watch or Samsung Gear; chest strap heart rate monitor 112; a sensor associated with mobile device 114 or exercise equipment 116 of FIG. 1, etc.) during presentation of the media asset (e.g., media asset 108 of FIG. 1). Based on the received input, the control circuity may determine biometric data of a user (e.g., a heart rate measurement, blood oxygen level measurement, respiratory rate, body temperature, etc.) during specific portions of an exercise. The control circuitry may compare the biometric data of the user to that of other users of a similar demographic and/or body type and/or physical fitness level, to determine whether the biometric data is within an acceptable range. In some embodiments, the control circuitry may generate a biometric data score indicative of a degree of difference of the user's biometric data from an acceptable range of biometric data.

At 804, control circuitry (e.g., control circuitry 504 of FIG. 5 and/or control circuitry 611 of FIG. 6) may receive input from one or more sensors (e.g., camera 114) indicative of posture or form of the user (e.g., user 102) during presentation of the media asset (e.g., media asset 108). For example, the control circuitry may utilize a stereo camera system or a camera capable of capturing images from multiple angles to generate a 3D representation of user 102 in a Cartesian coordinate system (e.g., 300 of FIG. 3). The control circuitry may compare the identified form or posture of the user during a specific exercise (e.g., a plank) to the form of the virtual instructor (e.g., virtual instructor 118 of FIG. 1, virtual instructor 302 of FIG. 3) and/or other users (e.g., having a rating of “Advanced” or otherwise determined to have proper posture or form during previous performances of the same workout). Based on this comparison, the control circuitry may generate a posture or form score indicative of a degree of difference from the optimal form for particular exercises. In the example of FIG. 3, the control circuitry may determine that the position of the upper back portion of the user is not proper since the user is arching his back, which may not sufficiently engage the abdominals in the workout and may put undue pressure on the arms and/or spine. Such determination may negatively impact the posture of form score assigned by the control circuitry to the user (e.g., user 102 of FIGS. 1, 3).

At 806, control circuitry (e.g., control circuitry 504 of FIG. 5 and/or control circuitry 611 of FIG. 6) may receive information provided by the user (e.g., user 102) related to an injury. For example, the user (e.g., user 102 of FIG. 1) may specify a particular injury he or she is suffering from. The control circuitry may reference a database (e.g., database 605 of FIG. 6), which may store a profile of the user and/or which may store anonymized data of other users indicating injuries such other users have suffered from and exercises that likely caused the injury. Based on the information retrieved from the database, the control circuitry may select a suitable workout that is less likely to aggravate the injury of the user. In some embodiments, the control circuitry may assign higher weights or scores to alternate versions of an exercise that are not associated with an injury similar to the injury reported by the user.

At 808, control circuitry (e.g., control circuitry 504 of FIG. 5 and/or control circuitry 611 of FIG. 6) may receive information related to nutrition and/or other habits of the user. For example, the control circuitry may receive (e.g., via the media application, wearable device, or other application interfacing with the media application) information indicative of nutrition (e.g., calories, proteins, carbohydrates, water, etc.), sleep (e.g., hours of sleep the user had in the past 24 hours or past few days), amount of time and intensity of recent workouts (e.g., which may increase the likelihood the user is sore), amount of steps the user has taken in the past day or past few days, etc. Based on this data, the control circuitry may compute a nutrition or current state score. For example, this score may be computed based on referencing a database (e.g., database 605 of FIG. 6) storing a profile of the user and other users, to compare how the user, or other similar users, fared in similar workouts with similar levels of nutrition consumed and/or sleep levels and/or when certain workouts were performed recently.

At 810, control circuitry (e.g., control circuitry 504 of FIG. 5 and/or control circuitry 611 of FIG. 6) may implement a prediction module, which may take as input each of the computed characteristics or scores from 802, 804, 806, 808. In some embodiments, an intensity-determining algorithm may consider the scores in combination (e.g., by computing an average among the scores, and/or identifying a highest and lowest score) when determining whether an alternate version of a current or future exercise should be provided. For example, the control circuitry may determine a success rate of providing an alternate workout in prior situations (for the current user and/or other users) with similar computed scores, and determine whether to provide the alternate version of the workout based on such success rate. In some embodiments, if each of the scores falls within an acceptable range (e.g., within 10 points of a threshold score) or the average of the scores falls within an acceptable range (e.g., within 5 points of a threshold score), the control circuitry may determine at 812 that an alternate version of the current or a future exercise should not be provided. On the other hand, if any of the scores fall outside the acceptable range and/or the average falls outside the acceptable range, the control circuitry may determine that an alternate version of the exercise should be provided.

In some embodiments, prediction module 810 may comprise a machine learning model, e.g., a neural network, that is trained to take as input various scores or characteristics represented in vector form (e.g., associated with biometric data, posture or form, a likelihood that a workout may aggravate an injury, nutrition or lifestyle habits) and output a probability that an alternate version of a workout should be provided. For example, the control circuitry may train the machine learning model using labeled training data (e.g., a series of scores or characteristics in vector form) paired with an indication of whether the determination of whether to provide an alternate version of an exercise was successful (e.g., based on user feedback and/or measured performance of the user of the training example). Thus, the machine learning model may be trained to recognize certain patterns of input data as predictive of whether an alternate exercise should be provided. At 812, the control circuitry may determine whether the alternate version of the exercise or workout should be provided based on the probabilities output by the trained machine learning model.

The processes discussed above are intended to be illustrative and not limiting. One skilled in the art would appreciate that the steps of the processes discussed herein may be omitted, modified, combined and/or rearranged, and any additional steps may be performed without departing from the scope of the invention. More generally, the above disclosure is meant to be exemplary and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods.

What is claimed is:

1. A method comprising:
generating for presentation to a user a media asset comprising a plurality of segments, wherein the media asset when generated for presentation depicts one or more exercises, and each respective exercise corresponds to one or more segments of the plurality of segments;
receiving input from one or more sensors during the presentation of the media asset, wherein the input is related to the user;
determining, based on the received input, that an alternate version of an exercise corresponding to at least one particular segment of the plurality of segments should be provided instead of a version of the exercise scheduled to be provided;
modifying the at least one particular segment to correspond to the alternate version of the exercise; and
generating for presentation to the user the at least one modified particular segment.

2. The method of claim 1, wherein at least one of the one or more sensors is associated with exercise equipment used during the exercise.

3. The method of claim 1, further comprising:
receiving user information from a plurality of users, wherein the user information is associated with exercise sessions that respective users of the plurality of users participated in; and
identifying, based on the received user information, a particular exercise of the exercise sessions associated with a user injury,
wherein the alternate version of the exercise is provided based on the received input from the one or more sensors and the identified exercise associated with the user injury.

4. The method of claim 1, wherein:
determining that the alternate version of the exercise corresponding to the at least one particular segment should be provided comprises:
determining, based on the input from one or more sensors, that an additional user, in addition to the user, is consuming the media asset; and
determining a current state of the user and the additional user based on the input from one or more sensors;
the alternate version of the exercise corresponds to a joint exercise for each of the user and the additional user; and
a recommendation is provided, based on the determined current states of the user and the additional user, that either the user or the additional user perform a higher intensity portion of the joint exercise than the other of the user or the additional user.

5. The method of claim 1, wherein:
each respective exercise is associated with a plurality of versions of the exercise corresponding to different intensity levels of the exercise, each version of the exercise being tagged with an indication of one or more attributes;
determining, based on the received input, that the alternate version of the exercise corresponding to the at least one particular segment should be provided comprises:
determining, based on the input from the one or more sensors, attributes associated with activity of the user during the presentation of the at least one particular segment; and
comparing the determined attributes to the tagged attributes to determine a version of the exercise having attributes matching the determined attributes; and
modifying the at least one particular segment to correspond to the alternate version of the exercise is performed based on the comparing.

6. The method of claim 1, wherein:
the received input corresponds to biometric data of the user;
determining, based on the received input, that the alternate version of the exercise corresponding to the at least one particular segment should be generated for presentation comprises:
determining, based on the received biometric data, a current state of the user during a current segment of the media asset; and
determining, based on the current state of the user, that the alternate version of the exercise should be provided during a next segment of the media asset following the current segment.

7. The method of claim 6, wherein:
each respective exercise is associated with a plurality of versions of the exercise corresponding to different intensity levels of the exercise; and
determining, based on the current state of the user, that the alternate version of the exercise should be provided during the next segment of the media asset following the current segment comprises:
determining that the received biometric data of the user is outside a predefined range; and
in response to determining that the received biometric data of the user is outside the predefined range, causing the alternate version of the exercise to be a lighter intensity version compared to the version of the exercise scheduled to be provided.

8. The method of claim 6, further comprising:
receiving nutrient information related to nutrients consumed by the user within a predefined period of time from a current time,
wherein the current state of the user during the current segment of the media asset is determined based on the received biometric data of the user and the received nutrient information.

9. The method of claim 6, further comprising:
identifying, based on the received input, a posture or form of the user during the current segment;
determining whether the identified posture or form of the user is proper; and
determining the current state of the user during the current segment of the media asset is based on the received biometric data of the user and whether the identified posture or form of the user is proper.

10. The method of claim 6, wherein:
the biometric data of the user is received during a warm-up segment of the media asset used to assess the physical abilities of the user.

11. A system comprising:
memory configured to store a media asset;
control circuitry configured to:
generate for presentation to a user the media asset comprising a plurality of segments, wherein the media asset when generated for presentation depicts one or more exercises, and each respective exercise corresponds to one or more segments of the plurality of segments;
receive input from one or more sensors during the presentation of the media asset, wherein the input is related to the user;
determine, based on the received input, that an alternate version of an exercise corresponding to at least one particular segment of the plurality of segments should be provided instead of a version of the exercise scheduled to be provided;
modify the at least one particular segment to correspond to the alternate version of the exercise; and
generate for presentation to the user the at least one modified particular segment.

12. The system of claim 11, wherein at least one of the one or more sensors is associated with exercise equipment used during the exercise.

13. The system of claim 11, wherein the control circuitry is further configured to:
receive user information from a plurality of users, wherein the user information is associated with exercise sessions that respective users of the plurality of users participated in; and
identify, based on the received user information, a particular exercise of the exercise sessions associated with a user injury,
provide the alternate version of the exercise based on the received input from the one or more sensors and the identified exercise associated with the user injury.

14. The system of claim 11, wherein:
the control circuitry is configured to determine that the alternate version of the exercise corresponding to the at least one particular segment should be provided by:
determining, based on the input from one or more sensors, that an additional user, in addition to the user, is consuming the media asset; and
determining a current state of the user and the additional user based on the input from one or more sensors;
the alternate version of the exercise corresponds to a joint exercise for each of the user and the additional user; and
the control circuitry is further configured to provide a recommendation, based on the determined current states of the user and the additional user, that either the user or the additional user perform a higher intensity portion of the joint exercise than the other of the user or the additional user.

15. The system of claim 11, wherein:
each respective exercise is associated with a plurality of versions of the exercise corresponding to different intensity levels of the exercise, each version of the exercise being tagged with an indication of one or more attributes;
the control circuitry is configured to determine, based on the received input, that the alternate version of the exercise corresponding to the at least one particular segment should be provided by:
determining, based on the input from the one or more sensors, attributes associated with activity of the user during the presentation of the at least one particular segment; and
comparing the determined attributes to the tagged attributes to determine a version of the exercise having attributes matching the determined attributes; and
the control circuitry is configured to modify the at least one particular segment to correspond to the alternate version of the exercise based on the comparing.

16. The system of claim 11, wherein:
the received input corresponds to biometric data of the user;
the control circuitry is configured to determine, based on the received input, that the alternate version of the exercise corresponding to the at least one particular segment should be generated for presentation by:
determining, based on the received biometric data, a current state of the user during a current segment of the media asset; and
determining, based on the current state of the user, that the alternate version of the exercise should be provided during a next segment of the media asset following the current segment.

17. The system of claim 16, wherein:
each respective exercise is associated with a plurality of versions of the exercise corresponding to different intensity levels of the exercise; and
the control circuitry is configured to determine, based on the current state of the user, that the alternate version of the exercise should be provided during the next segment of the media asset following the current segment by:
determining that the received biometric data of the user is outside a predefined range; and
in response to determining that the received biometric data of the user is outside the predefined range, causing the alternate version of the exercise to be a lighter intensity version compared to the version of the exercise scheduled to be provided.

18. The system of claim 16, wherein the control circuitry is further configured to:
receive nutrient information related to nutrients consumed by the user within a predefined period of time from a current time,
determine the current state of the user during the current segment of the media asset based on the received biometric data of the user and the received nutrient information.

19. The system of claim 16, wherein the control circuitry is further configured to:
identify, based on the received input, a posture or form of the user during the current segment;
determine whether the identified posture or form of the user is proper; and
determine the current state of the user during the current segment of the media asset based on the received biometric data of the user and whether the identified posture or form of the user is proper.

20. The system of claim 16, wherein:
the biometric data of the user is received during a warm-up segment of the media asset used to assess the physical abilities of the user.
